(19) United States
(12) Patent Application Publication
(10) Pub. No.: US 2011/0087249 A1
(43) Pub. Date: Apr. 14, 2011

(54) Internal Tissue Anchors

(75) Inventors: Anibal Rodrigues, Milford, CT (US); Scott DePierro, Madison, CT (US)

(73) Assignee: Tyco Healthcare Group LP, North Haven, CT (US)

(21) Appl. No.: 12/896,946

(22) Filed: Oct. 4, 2010

Related U.S. Application Data
(60) Provisional application No. 61/250,074, filed on Oct. 9, 2009.
(60) Provisional application No. 61/250,072, filed on Oct. 9, 2009.

(51) Int. Cl. A61B 17/00

(52) U.S. Cl. 606/151

(57) ABSTRACT

An internal tissue anchor is provided for retracting tissue and/or organs during a surgical procedure. The anchor includes a first magnet coupled to soft tissue and a second magnet coupled to a desired tissue or organ. The second magnet retracts the desired tissue or organ when magnetically coupled to the first magnet.

### CROSS REFERENCE TO RELATED APPLICATIONS

The present application claims priority to U.S. Provisional Application Ser. No. 61/250,074, filed Oct. 9, 2009, entitled “INTERNAL TISSUE ANCHORS” and U.S. Provisional Application Ser. No. 61/250,072, filed Oct. 9, 2009, entitled “MESH RETRACTORS WITH ADJUSTERS”, the contents of which are hereby incorporated by reference in their entirety.

### BACKGROUND

1. Technical Field

The present disclosure relates to systems and devices for retracting organs and/or body tissue during surgical procedures and, more particularly, to internal tissue anchors for retracting or positioning body tissue and/or body organs during minimally invasive surgery.

2. Background of the Related Art

As a result of the recent technological improvements in surgical instruments, surgical procedures, using minimally invasive techniques (e.g., endoscopic, laparoscopic, etc.), are routinely performed that cause less trauma to the patient.

In endoscopic and laparoscopic surgical procedures, it is often necessary to provide instrumentation to move or manipulate tissue and/or organs located in the area of operation. Generally, laparoscopic surgical procedures involve the introduction of a gas, such as, carbon dioxide, to insufflate a body cavity, e.g., the abdomen, to provide a working area for the surgeon. A trocar device is utilized to puncture the peritoneum to provide an access port by way of a cannula through the abdominal wall for the introduction of surgical instrumentation. After puncturing the peritoneum, the abdomen is insufflated. Generally, a trocar/cannula is placed through the abdominal wall for each piece of surgical instrumentation which is necessary to carry out the surgical procedure. In this manner, the surgeon may view the surgical site through an endoscope provided through a first trocar/cannula, and utilize a second trocar/cannula to introduce a surgical instrument such as a grasper, scissor, clip applier, stapler and any other surgical instrument which may be necessary during the particular surgical procedure.

Although the insufflation gas expands the abdomen to permit the surgeon to view the surgical site, it is often necessary to manipulate the internal organ or tissues to provide a clear path to the surgical objective. In the past, grasping tools have been utilized which pull on the organ or tissues to move them out of the way to provide a clear visual path for the surgeon. Endoscopic retractor mechanisms also have been developed which are utilized to push and hold the tissue or organ away from the surgical site.

Such grasping tools and retractor mechanisms have a disadvantage in that the surgeon operating the tools is required to use one hand to operate the grasping tool or retractor mechanism while using their other hand to perform the surgical procedure. Accordingly, a need exists for an internal tissue retractor that retracts and maintains tissue and/or organs in a retracted position while allowing a surgeon to use both hands during a surgical procedure.

Surgeons employ the use of anchors that are affixed internally to soft tissue. One such anchor uses magnets to retract tissue or organ. In such a method, a first magnet is attached to tissue within a body cavity and a second magnet is attached outside of the body. The second magnet interacts with the first magnet to move tissue or organ out of the way during surgery. Such a method has a drawback in that, for obese patients, the magnets may not have sufficient attractive force to interact effectively.

Alternatively, anchors can be fastened internally to soft tissue, e.g., an abdominal wall, during the surgical procedure using hooks or other sharp objects. During a surgical procedure, it may be necessary to move the location of the anchors to obtain a better view of body cavity in order to perform the surgical procedure. The use of such hooks and other sharp objects may cause greater trauma to a patient by removing the hooks.

Further, during a surgical procedure, a surgeon may fail to remove the anchor from the body cavity. In these instances, the anchor remains in the body cavity and may cause health issues such as infection. Further, a surgeon may have to reenter the body cavity to remove the anchor thereby causing further trauma to the patient.

### SUMMARY

The present disclosure relates to internal tissue anchors used to retract tissue or organs.

In an embodiment of the present disclosure, an internal tissue anchor is provided having at least one attachment device configured to attach the internal tissue anchor to an abdominal wall, a first magnet coupled the attachment device and a second magnet coupled to a desired tissue or organ. The second magnet retracts the desired tissue or organ when magnetically coupled to the first magnet. In the embodiment, the attachment device may be at least one hook, a suture, a clip, a helical coil, an I-clip, a spike or a tack. The second magnet may be coupled to a tissue clip, a hook or a wire loop.

Alternatively, the first magnet may have a first end coupled to a first attachment device affixed to the abdominal wall and a second end coupled to a second attachment device affixed to the abdominal wall. The second magnet can be moved to any position between the first end and second end of the first magnet.

In another embodiment of the present disclosure, an internal tissue anchor includes a retractable hook operable to engage a soft tissue, a chamber configured to receive the retractable hook and a distal end configured to suspend a desired tissue or organ. The retractable hook is formed from a shape memory alloy so that when the retractable hook is retracted into the chamber the retractable hook is straightened and when the retractable hook is released from the chamber the retractable hook regains its hook shape. The distal end may include a magnet configured to attract a magnet of opposing polarity coupled to the desired tissue or organ. Alternatively, the distal end may have a protrusion with an aperture where the aperture is configured to receive a hook coupled to the desired tissue or organ.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 depicts an internal tissue anchor in accordance with an embodiment of the present disclosure;

FIGS. 2A through 2F depict internal tissue anchors in accordance with an embodiment of the present disclosure;

FIGS. 3A and 3B depict internal tissue anchors in accordance with an embodiment of the present disclosure;

FIG. 4 depicts an internal tissue anchor in accordance with another embodiment of the present disclosure;

FIGS. 5A through 5D depict internal tissue anchors in accordance with another embodiment of the present disclosure;

FIG. 6A through 6D depict internal tissue anchors in accordance with another embodiment of the present disclosure;

FIGS. 7A and 7B depicts internal tissue anchors in accordance with another embodiment of the present disclosure; and

FIGS. 8A-8C depict tissue attachment devices according to embodiments of the present disclosure.

### DETAILED DESCRIPTION

Particular embodiments of the present disclosure are described hereinbelow with reference to the accompanying drawings; however, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. Like reference numerals may refer to similar or identical elements throughout the description of the figures.

As used herein, the term “distal” refers to that portion of the instrument, or component thereof which is farther from the user while the term “proximal” refers to that portion of the instrument or component thereof which is closer to the user.

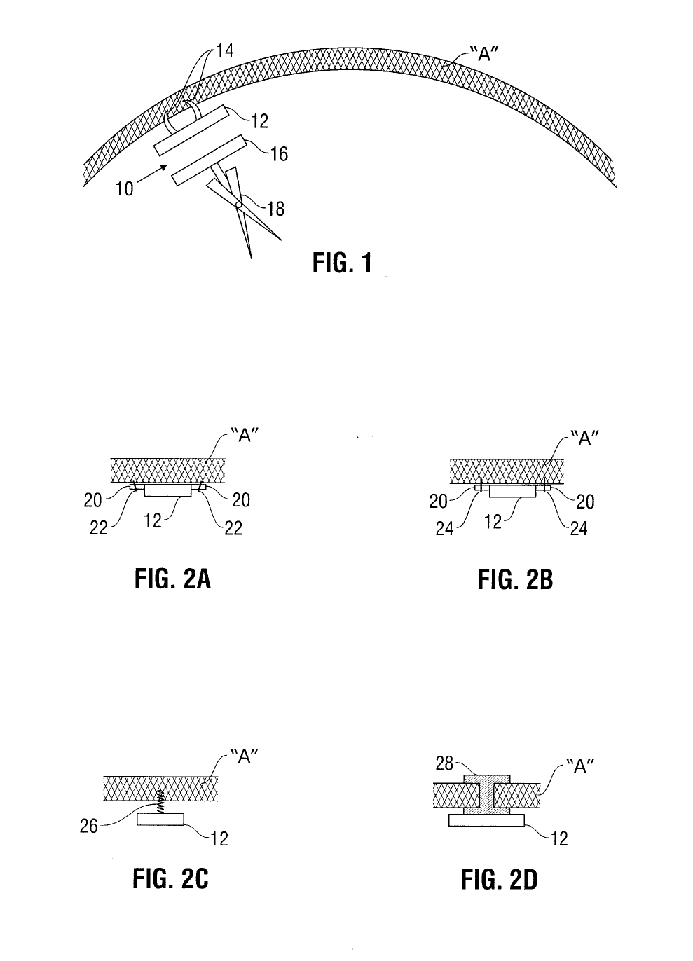

FIG. 1 depicts an internal tissue anchor 10 in accordance with an embodiment of the present disclosure. As shown in FIG. 1, anchor 10 is affixed to soft tissue, e.g., abdominal wall “A” and has a first magnet 12 affixed to abdominal wall “A” and a second magnet 16. Magnet 12 can be affixed to abdominal wall “A” using a variety of methods. For instance, as shown in FIG. 1, a pair of opposing hooks 14 are used to secure magnet 12 to abdominal wall “A”. Hooks 14 can be made from any engineering metals and/or plastics that would support tissue anchor 10 and any tissue or organ retracted therefrom and is suitable for use in a body cavity. Second magnet 16 has a tissue clip 18 attached thereto to retract tissue or organ. Tissue clip 18 can be made from stainless steel or any other engineering metals and/or plastic suitable for use in a body cavity.

Magnet 12 and magnet 16 can be made from ferromagnetic materials which include iron ore (magnetite or lodestone), cobalt and nickel, as well the rare earth metals gadolinium and dysprosium. Magnets 12 and 16 may also be made from composite materials such as ceramic or ferrite, alnico (a combination of aluminum, nickel and cobalt with iron) or ticonal (a combination of titanium, cobalt, nickel and aluminum with iron). Magnet 12 and magnet 16 have different polarities so that they attract each other.

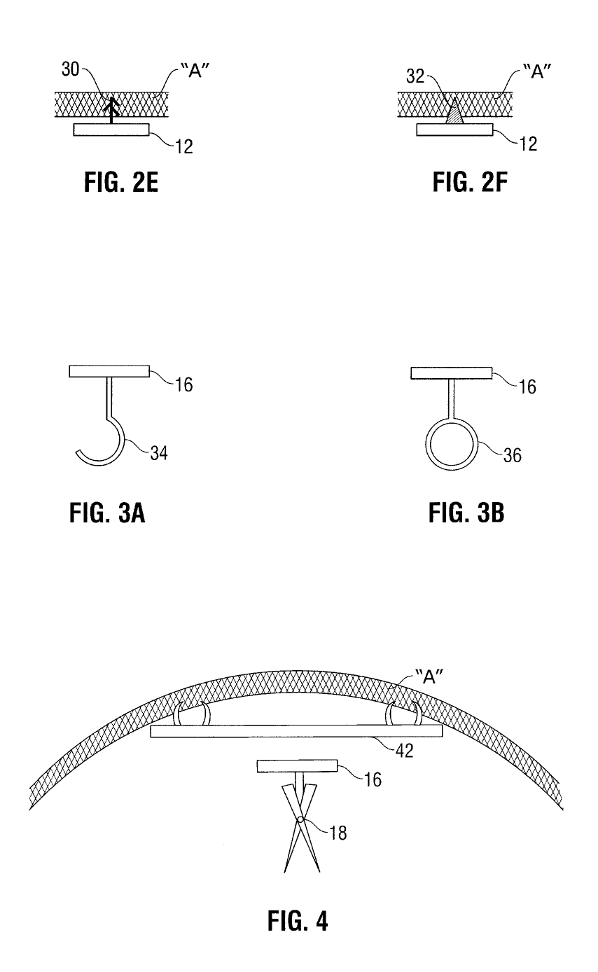

FIGS. 2A through 2F depict alternative methods for affixing magnet 12 to abdominal wall “A”. As shown in FIG. 2A, magnet 12 has a pair of extensions 20 that rest along abdominal wall “A”. Sutures 22 are applied to extensions 20 in order to secure magnet 12 to abdominal wall “A”. As shown in FIG. 2B, a clip or staple 24 may be utilized to secure magnet 12 by securing extensions 20 to abdominal wall “A”. FIG. 2C shows a helical coil 26 coupled to magnet 12. Helical coil 26 may be formed from titanium or any other engineering metals and/or plastic suitable for use in a body cavity. FIG. 2D depicts an I-clip 28 coupled to magnet 12 which extends through abdominal wall “A”. FIG. 2E depicts a spike 30 coupled to magnet 12 which is driven into abdominal wall “A”. FIG. 2F depicts a tack 32 coupled to magnet 12 for securing the magnet 12 in abdominal wall “A”.

Although FIG. 1 depicts a tissue clip 18 attached to magnet 16, other attachments may be used to retract tissue and/or organ. For instance, as shown in FIG. 3A, hook 34 is coupled to magnet 16. Hook 34 is used to keep tissue or organ in the retracted position. Alternatively, as shown in FIG. 3B, a wire loop 36 may be used to keep tissue or organ in the retracted position. Wire loop 36 can be made from stainless steel, titanium, or any other engineering materials and/or plastics suitable for use in a body cavity. Hooks, slings or wires (not shown) may be coupled to the tissue and/or organs to suspend the tissue and/or organs from hook 34 or wire loop 36.

FIG. 4 depicts another tissue anchor according to an embodiment of the present disclosure. As shown in FIG. 4, a magnetic bar 42 is provided and affixed to abdominal wall “A” using any of the methods described above in regard to FIGS. 1 through 2F. Although magnetic bar 42 is shown to be affixed to abdominal wall “A” at two different locations, three or more locations may be used to affix the magnetic bar at different locations. More locations would allow a bigger magnetic bar to be attached to abdominal wall “A”. By using a magnetic bar 42, magnet 16 can be placed in any position along the magnetic bar 42. This would allow a clinician to easily move the position of the retracted tissue or organ without causing additional trauma to the patient.

During a surgical procedure, after a trocar pierces the peritoneum and prior to insufflating the abdomen, anchor 10 or magnetic bar 42 is affixed to abdominal wall “A” by one of the methods described above with regard to FIGS. 1-2F. Then a user or surgeon uses the second magnet 16 with an attachment for retracting tissue and/or organs such as the tissue clip 18 or a hook, sling and/or wire coupled to hook 34 or wire 36. Tissue and/or organs are attached to the attachment and the surgeon suspends the attachment by magnetically coupling magnet 12 and magnet 16. In the example involving a magnetic bar 42, a surgeon can reposition the retracted tissue and/or organs by sliding magnet 16 along the magnetic bar 42.

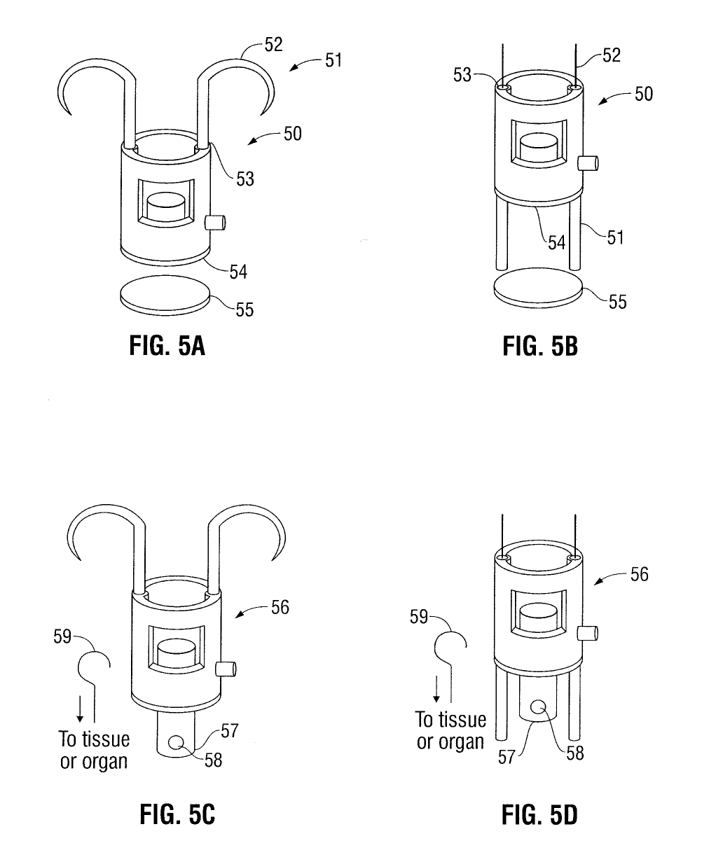

FIGS. 5A and 5B depict another internal tissue anchor in accordance with an embodiment of the present disclosure. An anchor 50 is provided that can be attached to abdominal wall “A”. Anchor 50 has least two wire hooks 51 made from Nitinol or any other shape memory alloy. Shape memory alloys undergo large deformation under stress, yet regain their intended shape once the metal is unloaded again. As shown in FIG. 5A, hook 51 has a proximal end 52 used to affix the anchor 50 to abdominal wall “A”. When a surgeon pulls hook 51 down, proximal end 52 is straightened and retracted into anchor 50 in chamber 53 (FIG. 5B). If the anchor is affixed to the abdominal wall “A”, pulling down on the hook releases the anchor from the abdominal wall “A”. Chamber 53 applies a force to proximal end 52 keeping it straight. When hook 51 is pushed upward such that chamber 53 no longer applies a force to end 52, hook 51 regains its intended shape as shown in FIG. 5A. This drives the hook into abdominal wall “A” thereby securing the anchor therein. A magnet 54 is affixed to the distal end of anchor 50. Magnet 54 has an opposite polarity than that of magnet 55. Magnet 55 is similar to magnet 16 described above and may have a tissue clip, a hook or a wire loop as shown in FIGS. 1, 3A and 3B to retract tissue or organ as described above.

FIGS. 5C and 5D depict an anchor 56 similar to anchor 50 shown in FIGS. 5A and 5B. Anchor 56 has a protrusion 57 at the distal end of anchor 56. Protrusion 57 includes an aperture or opening 58 configured to receive a hook 59 from which tissue or organ is suspended.

In another embodiment of the present disclosure, an anchor may be provided that is made from a material that is easily absorbable. Absorbable materials are broken down in tissue after a given period of time, which depending on the material, can be from ten days to eight weeks. They are used therefore in many of the internal tissues of the body.

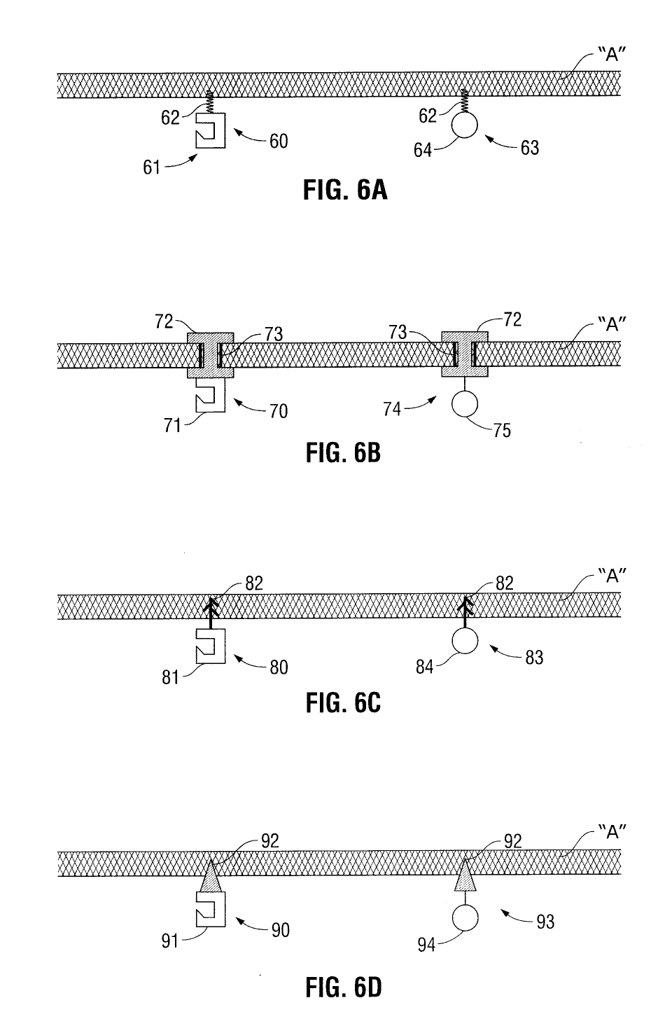

FIG. 6A depicts an anchor 60 in accordance with an embodiment of the present disclosure. Anchor 60 has a hook shaped distal end 61 and is made from a material that is easily absorbable. Hook shaped distal end 61 is coupled to a helical coil 62, such as Protack titanium helical coil, which is affixed to abdominal wall “A”. Alternatively, an anchor 63 may be provided where a looped suture 64 made from an absorbable material is coupled to helical coil 62. Hook shaped distal end 61 and looped suture 64 are configured to receive a hooking mechanism (not shown) attached to a tissue clamp that retracts the tissue or organ.

FIG. 6B depicts an anchor 70 in accordance with an embodiment of the present disclosure. Anchor 70 has a hook shaped distal end 71 and is made from a material that is easily absorbable. Hook shaped distal end 71 is coupled to an I-clip 72, which is affixed to abdominal wall “A”. I-clip 72 is situated in abdominal wall “A” by making an aperture 73 in wall “A” and placing the I-clip 72 through aperture 73. Alternatively, an anchor 74 may be provided where a looped suture 75 made from an absorbable material is coupled to I-clip 72. Hook shaped distal end 71 and looped suture 75 are configured to receive a hooking mechanism (not shown) attached to a tissue clamp that retracts the tissue or organ. I-clip 72 may be made from an absorbable material or non absorbable material.

FIG. 6C depicts an anchor 80 in accordance with an embodiment of the present disclosure. Anchor 80 has a hook shaped distal end 81 and is made from a material that is easily absorbable. Hook shaped distal end 81 is coupled to a spike 82, which pierces abdominal wall “A” and is secured therein. Spike 82 may be made from stainless steel, titanium or any other engineering materials and/or plastics suitable for use in a body cavity. Spike 82 may also be made from an absorbable material as described above. Alternatively, an anchor 83 may be provided where a looped suture 84 made from an absorbable material is coupled to spike 82. Hook shaped distal end 81 and looped suture 84 are configured to receive a hooking mechanism (not shown) attached to a tissue clamp that retracts the tissue or organ.

FIG. 6D depicts an anchor 90 in accordance with an embodiment of the present disclosure. Anchor 90 has a hook shaped distal end 91 and is made from a material that is easily absorbable. Hook shaped distal end 91 is coupled to a tack 92, which pierces abdominal wall “A” and is secured therein by twisting the tack in a circular motion. Tack 92 may be made from stainless steel, titanium or any other engineering materials and/or plastics suitable for use in a body cavity. Tack 92 may also be made from an absorbable material as described above. Alternatively, an anchor 93 may be provided where a looped suture 94 made from an absorbable material is coupled to tack 92. Hook shaped distal end 91 and looped suture 94 are configured to receive a hooking mechanism (not shown) attached to a tissue clamp that retracts the tissue or organ.

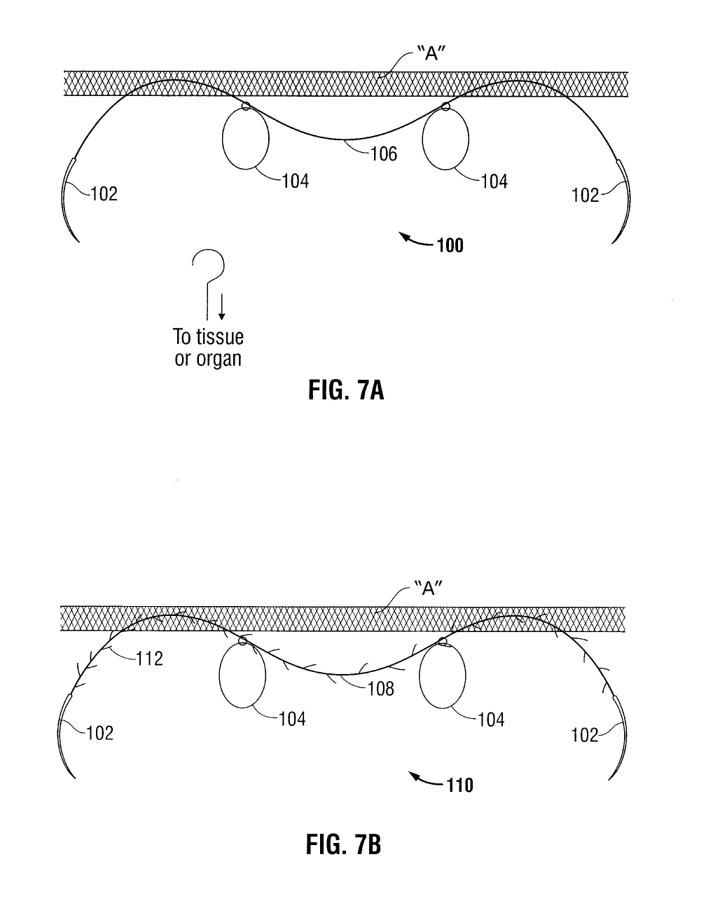

FIGS. 7A and 7B depict internal tissue anchor systems 100 and 110, respectively, in accordance with another embodiment of the present disclosure. System 100 includes a suture 106 that can be threaded through abdominal wall “A” using one of needles 102. Suture 106 may include at least one anchoring ring or loop 104 from which tissue or organs may be suspended using a hook or any other method described herein. Anchoring ring 104 may be coupled to suture 106 by any conventional means. Although FIG. 7A depicts a system 100 that includes two needles 102 and two anchoring rings 104, any number of needles 102 and corresponding anchoring rings 104 may be used. For instance, suture 106 may include a single thread having a single needle 102 with a single anchoring ring 104 or a suture 106 having multiple threads emanating from a single point where each thread has a corresponding needle 102 and anchoring ring 104 may be used. Suture 106 and anchoring rings 104 may be composed of standard suture material or an absorbable material that can be left inside the abdominal cavity thereby eliminating the worry of a non-absorbable device being lost or accidentally left in the abdominal cavity. Anchoring rings 104 may be disposed on suture 106 before suture 106 is disposed in the abdominal cavity or they may be added onto suture 106 after suture 106 is threaded into the abdominal wall “A”.

Alternatively, suture 106 may incorporate V-Loc technology as shown in FIG. 7B (suture 108). For instance, as shown in FIG. 7B, suture 108 includes unidirectional shallow barbs 112 that may have a circumferential distribution (not shown). Barbs 112 are evenly spaced throughout suture 108 to grasp the abdominal wall “A” at numerous points thereby spreading tension across the abdominal wall “A” and reducing trauma to the abdominal wall “A”.

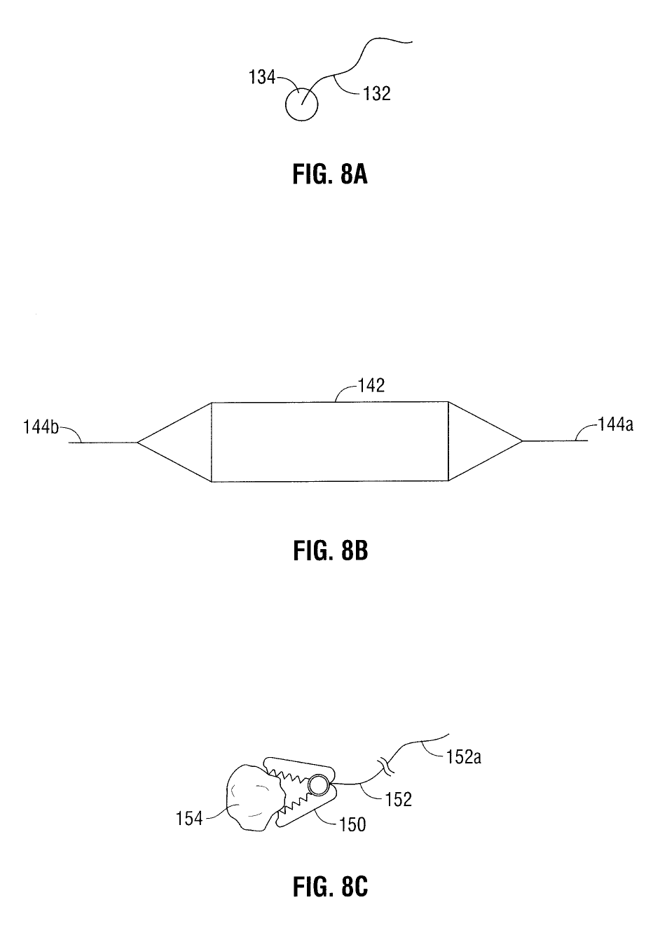

As described above, after a surgeon pierces the peritoneum and prior to insufflating the abdomen, the surgeon affixes at least one of the anchors described above to the abdominal wall “A” using one of the methods described above. Tissue and/or organs are attached to a tissue attachment device as shown in FIGS. 8A-8C. which is then suspended from the anchor. For example, as shown in FIG. 8A, a suture 132 may be provided that has a pledget 134 (e.g., a lcm diameter pledget) attached thereto. Pledget 134 may include an adhesive, barbs or any other means that may be used to attach pledget 134 to tissue and/or organs. The other end of suture 132 may be attached to one of the anchors described hereinabove. FIG. 8B depicts a sling 142 that may be used to suspend tissue and/or organs. The ends of sling 142 have suture leads 144a and 144b that are coupled to an anchor after the sling is placed beneath tissue and/or organs. FIG. 8C depicts an alligator clamp 150 at one end of suture 152. Alligator clamp 150 clamps on to tissue and/or or organ 154 to suspend the tissue and/or organ 154 from the abdominal wall. The proximal end 152a of suture 152 is connected to the anchor.

The anchors and tissue attachment devices described hereinabove with regard to FIGS. 6A-8C may be made from an absorbable material thereby eliminating the worry of a non-absorbable device being lost or accidentally left in the abdominal cavity.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figs. are presented only to demonstrate certain examples of the disclosure. Other elements, steps, methods and techniques that are insubstantially different from those described above and/or in the appended claims are also intended to be within the scope of the disclosure.

1. An internal tissue anchor comprising:
at least one attachment device configured to attach the internal tissue anchor to an abdominal wall;
a first magnet coupled to the attachment device; and
a second magnet coupled to a desired tissue or organ, the second magnet configured to retract the desired tissue or organ when magnetically coupled to the first magnet.

2. The internal tissue anchor according to claim 1, wherein the attachment device includes at least one hook.

3. The internal tissue anchor according to claim 1, wherein the attachment device includes a suture.

4. The internal tissue anchor according to claim 1, wherein the attachment device includes a clip.

5. The internal tissue anchor according to claim 1, wherein the attachment device includes a helical coil.

6. The internal tissue anchor according to claim 1, wherein attachment device includes an I-clip.

7. The internal tissue anchor according to claim 1, wherein the attachment device includes a spike.

8. The internal tissue anchor according to claim 1, wherein the attachment device includes a tack.

9. The internal tissue anchor according to claim 1, wherein the second magnet is coupled to a tissue clip configured to grasp the desired tissue or organ.

10. The internal tissue anchor according to claim 1, wherein the second magnet is coupled to a hook that is affixed to the desired tissue or organ.

11. The internal tissue anchor according to claim 1, wherein the second magnet is coupled to a wire loop that is affixed to the desired tissue or organ.

12. The internal tissue anchor according to claim 1, wherein the first magnet has a first end coupled to a first attachment device affixed to the abdominal wall and a second end coupled to a second attachment device affixed to the abdominal wall and wherein the second magnet can be moved to any position between the first end and second end of the first magnet.

13. An internal tissue anchor comprising:
a retractable hook operable to engage a soft tissue;
a chamber configured to receive the retractable hook; and
a distal end configured to suspend a desired tissue or organ.

14. The internal tissue anchor according to claim 13, wherein the retractable hook is formed from a shape memory alloy so that when the retractable hook is retracted into the chamber the retractable hook is straightened and when the retractable hook is released from the chamber the retractable hook regains its hook shape.

15. The internal tissue anchor according to claim 13, wherein the distal end comprises a first magnet configured to attract a second magnet of opposing polarity coupled to the desired tissue or organ.

16. The internal tissue anchor according to claim 13, wherein the distal end comprises a protrusion having an aperture, the aperture is configured to receive a hook coupled to the desired tissue or organ.
